(19) United States
(12) Patent Application Publication
(10) Pub. No.: US 2024/0393324 A1
(43) Pub. Date: Nov. 28, 2024

(54) Method for Magnetically Sorting Biological Objects

(71) Applicant: Applied Cells Inc., Santa Clara, CA (US)

(72) Inventors: Yuchen Zhou, San Jose, CA (US); Silin Sa, San Jose, CA (US); Liping Yu, San Jose, CA (US)

(21) Appl. No.: 18/795,047

(22) Filed: Aug. 5, 2024

Related U.S. Application Data
(63) Continuation-in-part of application No. 18/396,515, filed on Dec. 26, 2023.
Application No. 18/396,515 is a continuation-in-part of application No. 18/144,447, filed on May 8, 2023.
Application No. 18/144,447 is a continuation-in-part of application No. 18/111,486, filed on Feb. 17, 2023.
Application No. 18/111,486 is a continuation-in-part of application No. 18/072,362, filed on Nov. 30, 2022.
Application No. 18/072,362 is a continuation-in-part of application No. 16/729,398, filed on Dec. 29, 2019, now Pat. No. 11,571,696.
Application No. 16/729,398 is a continuation-in-part of application No. 15/911,115, filed on Mar. 3, 2018.
(60) Provisional application No. 63/406,437, filed on Sep. 14, 2022.

(51) Int. Cl. G01N 33/543; B82Y 15/00; G01N 33/569; G01N 33/58

(52) U.S. Cl. CPC G01N 33/54333; G01N 33/56972; G01N 33/585; B82Y 15/00; G01N 2333/70517; G01N 2333/70596

(57) ABSTRACT

A method for magnetically sorting biological objects comprising the steps of providing a fluid sample containing an initial biological mixture that includes magnetically labeled biological objects; depositing a first biological mixture on the conduit wall of a conduit exposed to a magnetic field by flowing the fluid sample through the conduit; rinsing the first biological mixture by flowing a stream of buffer fluid through the conduit and filling the conduit with the buffer fluid; removing the magnetic field from the conduit; resuspending the first biological mixture in the buffer fluid to form a suspension of the first biological mixture by applying mechanical agitation to the conduit; and depositing a second biological mixture on the conduit wall by applying the magnetic field to the suspension of the first biological mixture in the conduit, wherein the second biological mixture has a magnetically labeled biological object purity higher than the first biological mixture.

### CROSS-REFERENCE TO RELATED APPLICATIONS

The present application is a continuation-in-part of application Ser. No. 18/396,515, filed on Dec. 26, 2023, which is a continuation-in-part of application Ser. No. 18/144,447, filed on May 8, 2023, which is a continuation-in-part of application Ser. No. 18/111,486, filed on Feb. 17, 2023, which is a continuation-in-part of application Ser. No. 18/072,362, filed on Nov. 30, 2022, which claims priority to provisional application No. 63/406,437, filed on Sep. 14, 2022, and is a continuation-in-part of application Ser. No. 16/729,398, filed on Dec. 29, 2019, which is a continuation-in-part of application No. 15/911, 115, filed on Mar. 3, 2018. All of these applications are incorporated herein by reference in their entirety, including their specifications.

### BACKGROUND OF THE INVENTION

The present invention relates to separation and sorting of biological objects, and more particularly, to a method and apparatus for magnetically sorting biological objects.

The separation and sorting of biological objects or cells is critical to various biomedical applications, such as diagnostics and therapeutics. Biological objects may be sorted based on their respective physical properties, such as size and density, and biochemical properties, such as surface antigen expression.

In a biological object sorting process effectuated by an applied magnetic field, the biological object, such as a cell, which is typically nonmagnetic, can be magnetized for magnetic sorting purpose by attaching antibody-conjugated magnetic beads thereto, a process commonly known as magnetic labeling. FIG. 1A shows a cell 50 including a plurality of surface markers or antigens 52 on the cell surface thereof, and a plurality of antibody-conjugated magnetic beads 54 suspended in a fluid. Each of the antibody-conjugated magnetic beads 54 includes a magnetic entity 56 conjugated with one or more antibodies or other ligands 58, such as peptides and aptamers, that correspond to the surface markers 52. After an incubation period, the magnetic beads 54 may be directly attached to the cell 50 via the antigen-antibody interaction to form a magnetically labeled cell as shown in FIG. 1B, in a process known as direct labeling.

Alternatively, magnetic beads may be attached to a cell through an indirect labeling process. FIG. 2A shows a cell 50 including a plurality of surface markers or antigens 52 on the cell surface thereof, a plurality of intermediary links 60, and a plurality of magnetic beads 62 suspended in a fluid. Each of the intermediary links 60 includes one or more linking molecules 64, such as biotin or phycoerythrin (PE), conjugated to a primary antibody 66 that corresponds to the surface markers 52 of the cell 50. Each of the magnetic beads 62 includes a magnetic entity 56 conjugated with one or more secondary antibodies or ligands 68, such as streptavidin, that target the linking molecules 64. After an incubation period, the intermediary links 60 may attach to the cell 50 via the antigen-antibody interaction, and the magnetic beads 62 may further attach to the intermediary links 60 via PE-antibody, biotin-streptavidin, or other types of interactions, thereby forming a magnetically labeled cell as shown in FIG. 2B.

The magnetic beads 54 and 62 should ideally exhibit no magnetic moment in the absence of an applied magnetic field, thereby making the labeled cells indistinguishable from other biological objects in a cell suspension. As such, the magnetic entity 56 of the magnetic beads 54 and 62 normally consists of a magnetic nanoparticle or an aggregate of magnetic nanoparticles encapsulated in a nonmagnetic matrix because a magnetic particle may exhibit superparamagnetism as its size is reduced to tens of nanometers. In a sufficiently small ferromagnetic (e.g., iron) or ferrimagnetic (e.g., iron oxide) nanoparticle that exhibits superparamagnetism, magnetization can randomly flip direction under the influence of temperature. The typical time period between two such consecutive flips is known as the Neel relaxation time, or simply the relaxation time. Therefore, when the time period used to measure the magnetization of the magnetic nanoparticle is longer than the relaxation time thereof, the magnetic nanoparticle would appear to be nonmagnetic in the absence of an external magnetic field.

During the cell sorting process, the magnetic beads 54 and 62 attached to cells are first magnetized by sufficiently high magnetic field generated by a magnetic separator device and then attracted to regions of high magnetic field gradient. The size of commercially available magnetic beads can range from ˜50 nm (e.g., Miltenyi Biotec's MACS Microbeads) to several microns (e.g., Thermo Fisher's Dynabeads) in diameter. Larger magnetic beads have higher magnetic moment and can thus generate higher attraction force compared to smaller magnetic beads at a given magnetic field strength. However, excess large magnetic beads attached to a cell may potentially cause epitope blocking.

After cells in fluid sample are magnetically labeled, they can be sorted or separated from the other non-labeled cells or biological objects in the fluid sample by a magnetic separator device. FIG. 3A shows a conventional magnetic separator device 70 comprising a container vessel 72 for holding a static fluid sample 74, which contains the magnetically labeled cells 76 and non-labeled cells or biological objects 77, and a permanent magnet 78 placed in close proximity to a wall of the container vessel 72. The permanent magnet 78 generates a magnetic field in the container vessel 72 with the magnetic field gradient pointing towards the permanent magnet 78. After sufficient time, the magnetically labeled cells 76 will be gradually pulled by the force produced by the magnetic field towards the vessel wall and form an aggregate at the vessel wall, as shown in FIG. 3B. Because the magnetic field strength rapidly decreases as the distance from the permanent magnet 78 increases, the size of the vessel 72 and the fluid sample volume will be adversely limited. The weak magnetic field strength of the conventional magnetic separator device 70 requires the use of larger magnetic beads (e.g., greater than 100 nm in diameter).

FIG. 4 illustrates another conventional magnetic separator device 80 that separates magnetically labeled cells in static fluid sample contained in one or more wells 82. The magnetic device 80 uses multiple ferromagnetic poles 84, each of which has a trapezoidal tip, to act as a guide to concentrate the magnetic flux generated by multiple permanent magnets 86 attached thereto to increase the magnetic field strength and gradient near their tips. The corresponding magnetic field distribution, as delineated by magnetic field lines 88, shows that the magnetic field is strongest between the side surfaces of adjacent trapezoidal tips, as indicated by the small spacing between the field lines 88. By contrast, the magnetic field is much weaker above the pole tips, as indicated by the large spacing between the field lines 88. Accordingly, this necessitates the bottom portion of each well 82 to be disposed between the side surfaces of the pole tips, where the magnetic field is strong. The magnetically labeled cells in the conical-shaped wells 82 will be collected or condensed on or near the bottom of the wells 82 adjacent to the side surfaces of the trapezoidal tips of the ferromagnetic poles 84. Compared with the magnetic separator device 70 utilizing only the permanent magnet 78, the magnetic separator device 80 may improve the magnetic field strength and gradient by using the ferromagnetic poles 84 to concentrate the magnetic flux. Both devices 70 and 80, however, are designed to treat static fluid sample and thus may have limited throughput.

FIG. 5 illustrates a conventional magnetic separator device 90 that separates the magnetically labeled cells as a fluid sample flows through the device 90. The device 90 includes a chamber 92 disposed between a pair of permanent magnets 94 that generate a magnetic field 96 across the chamber 92. The chamber 92 is filled with a porous magnetic matrix comprising an aggregate of ferromagnetic spheres 98 that may be magnetized by the magnetic field 96 to produce relatively strong localized magnetic field and field gradient in small gaps between the ferromagnetic spheres 98, thereby magnetizing the magnetically labeled cells and attracting them to the surface of the ferromagnetic spheres 98 as the fluid sample is filtered through the chamber 92. The chamber 92 and the aggregate of ferromagnetic spheres 98 disposed therein are collectively referred to as a “magnetic column.” Compared with the magnetic beads attached to the magnetically labeled cells, the ferromagnetic spheres 98 are much larger and may produce remanent magnetization after the magnetic field 96 is removed from the chamber 92. The remanent magnetization may prevent or hinder the detachment of the magnetically labeled cells from the surface of the particles or spheres 98 even after the removal of the magnetic field 96. While the magnetic separator device 90 may operate in a continuous flow manner and thus may potentially have a higher throughput than the magnetic separator devices 70 and 80 that operate in a static manner, the recovery of the magnetically labeled cells in certain applications (e.g., positive selection process where the magnetically labeled cells are the target cells) may be lower even with vigorous flushing of the magnetic column using pressurized fluid to dislodge the magnetically labeled cells trapped in the magnetic column, which may damage the same cells. The higher local field strength and field gradient of the column-based magnetic separator device 90 allow the use of magnetic beads as small as 50 nm in diameter.

FIG. 6 shows another magnetic separator device 104, which operates in a continuous flow manner without using a magnetic column, thereby obviating the potential recovery issue associated therewith. The column-free device 104 includes a conduit 106 surrounded by a radial array of ferromagnetic poles 108 that conduct magnetic flux from a plurality of permanent magnets 110 and 112. Unlike the column-based magnetic separator device 90, the fluid sample flows through the conduit 106 unimpeded along a direction perpendicular to the figure. The magnetic separator device 104 essentially rearranges the linear array of the ferromagnetic poles 84 of the static magnetic separator device 80 in a radial manner to create a magnetic periodic field at the center of the radially arranged ferromagnetic poles 108 and permanent magnets 110 and 112. Like the static device 80 shown in FIG. 4, the corresponding magnetic field distribution generated by the device 104, as delineated by magnetic field lines 114 between the trapezoidal tips of the ferromagnetic poles 108, shows that the magnetic field is strongest between the side surfaces of adjacent trapezoidal tips, as indicated by the small spacing between the field lines 114, and much weaker above the pole tips (i.e., inside the conduit 106), as indicated by the large spacing between the field lines 114. However, unlike the wells 82 that extend into the regions between the side surfaces of two adjacent trapezoidal tips, the conduit 106 of the magnetic separator device 104 does not extend into such regions, thereby making the magnetic field in the conduit 106 considerably weaker. This is further exacerbated by the limited time exposed to the magnetic field as the fluid sample flows through the conduit 106.

For the foregoing reasons, there is a need for a method and apparatus for magnetically sorting biological objects that can efficiently extract and recover magnetically labeled target cells with high purity and recovery rate from a suspension of heterogeneous mixture of cells.

### SUMMARY OF THE INVENTION

The present invention is directed to a method that satisfies this need. A method having features of the present invention for magnetically sorting biological objects comprising the steps of providing a fluid sample containing an initial biological mixture that includes magnetically labeled biological objects and unlabeled biological objects; depositing a first biological mixture on a conduit wall of a conduit exposed to a magnetic field by flowing the fluid sample through the conduit; rinsing the first biological mixture by flowing a stream of buffer fluid through the conduit and filling the conduit with the buffer fluid; removing the magnetic field from the conduit containing the first biological mixture; resuspending the first biological mixture in the buffer fluid in the conduit to form a suspension of the first biological mixture by applying mechanical agitation to disperse the first biological mixture on the conduit wall into the buffer fluid; and depositing a second biological mixture on the conduit wall by applying the magnetic field to the conduit while the suspension of the first biological mixture in the conduit remains stagnant, wherein the second biological mixture has a magnetically labeled biological object purity higher than the first biological mixture.

### BRIEF DESCRIPTION OF DRAWINGS

These and other features, aspects, and advantages of the present invention will become better understood with regard to the following description, appended claims, and accompanying drawings where:

FIGS. 1A and 1B illustrate formation of a magnetically labeled cell by direct labeling process;

FIGS. 2A and 2B illustrate formation of a magnetically labeled cell by indirect labeling process;

FIGS. 3A and 3B illustrate sorting of magnetically labeled cells by a conventional static magnetic separator device;

FIG. 4 illustrates another conventional magnetic separator device for sorting

magnetically labeled cells in static fluid sample;

FIG. 5 illustrates a magnetic separator devices that utilize a chamber filled with a column

of porous aggregate of ferromagnetic or ferrimagnetic objects for sorting magnetically labeled cells flowing through the chamber;

FIG. 6 is a cross-sectional view corresponding to a column-free magnetic separator

device for sorting magnetically labeled cells flowing through a conduit;

FIG. 7 is a cross-sectional view of an exemplary magnetic separator device when the holder and conduit are disengaged from the magnetic assembly;

FIG. 8 is a cross-sectional view of the magnetic separator device of FIG. 7 when the conduit is squeezed against the tips of the magnetic assembly by the holder as the conduit engages the magnetic assembly;

FIG. 9 is a schematic diagram depicting an exemplary fluidic circuit including the

magnetic separator device when the holder and conduit are disengaged from the magnetic assembly;

FIG. 10 is a schematic diagram depicting the fluidic circuit including the magnetic separator device when the conduit engages the magnetic assembly;

FIG. 11A is a cross-sectional view illustrating deposition of magnetically labeled biological objects on the conduit wall as the fluid sample passes through the magnetic separator device;

FIG. 11B is a cross-sectional view illustrating dispersion of magnetically labeled

biological objects from the conduit wall into static or moving fluid by vibrating agitator arm;

FIG. 12 is a perspective view of an automated magnetic separator system that includes multiple fluidic circuits that can independently perform magnetic sorting;

FIG. 13 is a flow diagram illustrating selected steps for magnetically sorting biological objects in accordance with an embodiment of the present invention;

FIGS. 14A-14E are cross-sectional views illustrating various stages of a magnetic sorting process in accordance with an embodiment of the present invention;

FIG. 15 shows dot plots for initial MNC and standard samples, and final sample

processed in accordance with an embodiment of the present invention; and

FIG. 16 shows dot plots for initial PBMC and standard samples, and final sample processed in accordance with an embodiment of the present invention.

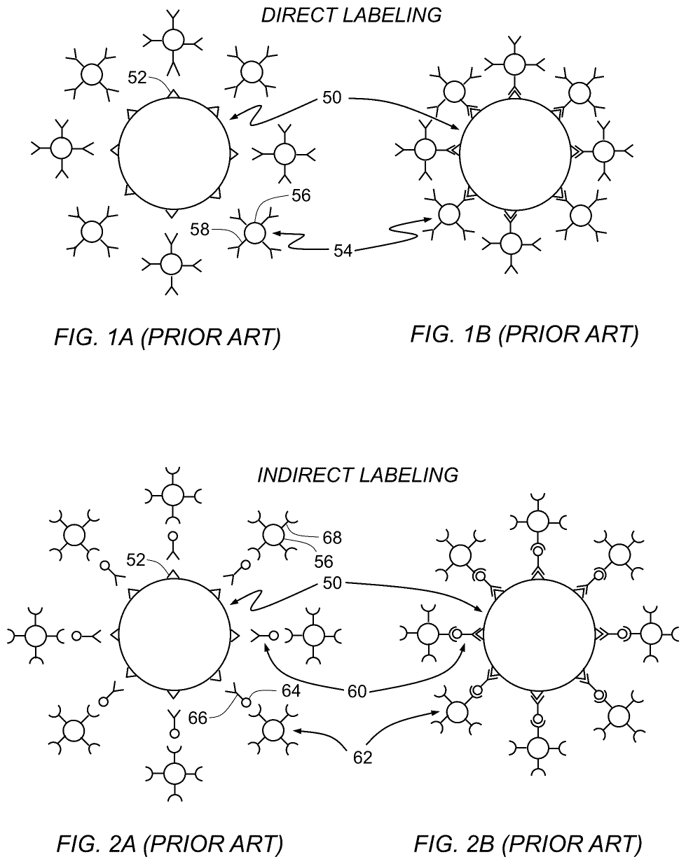

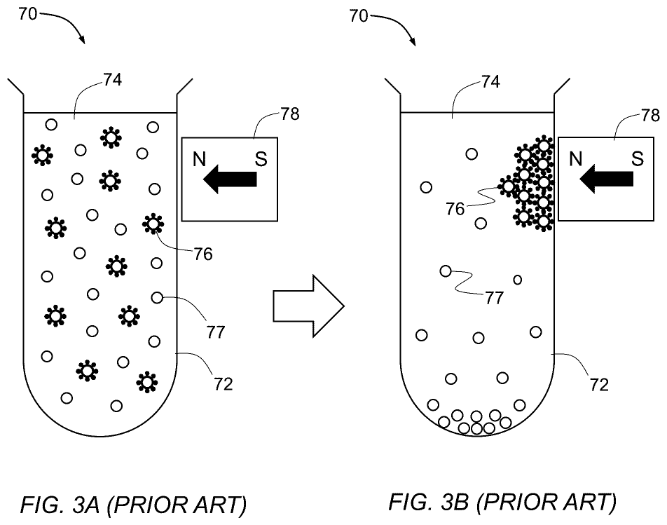

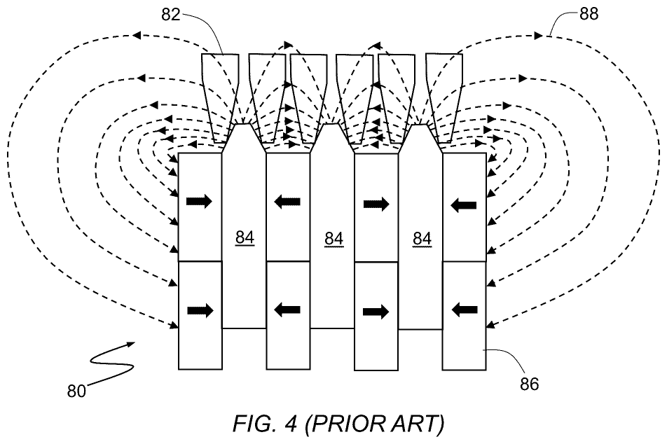

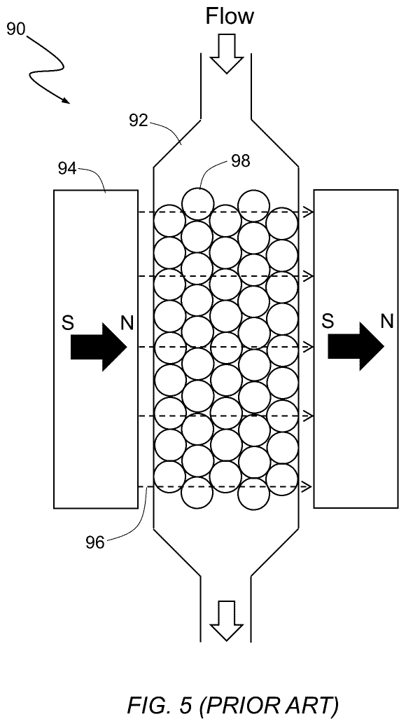

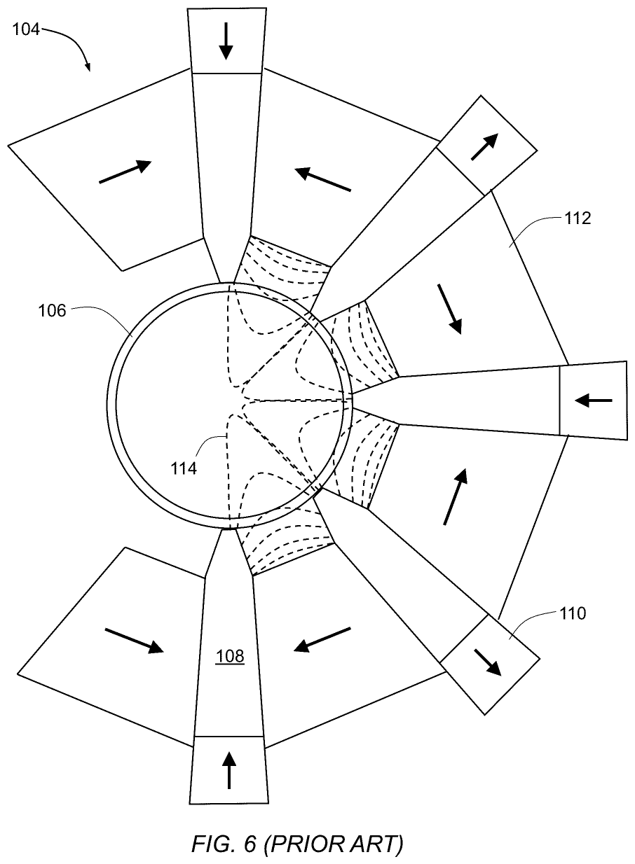

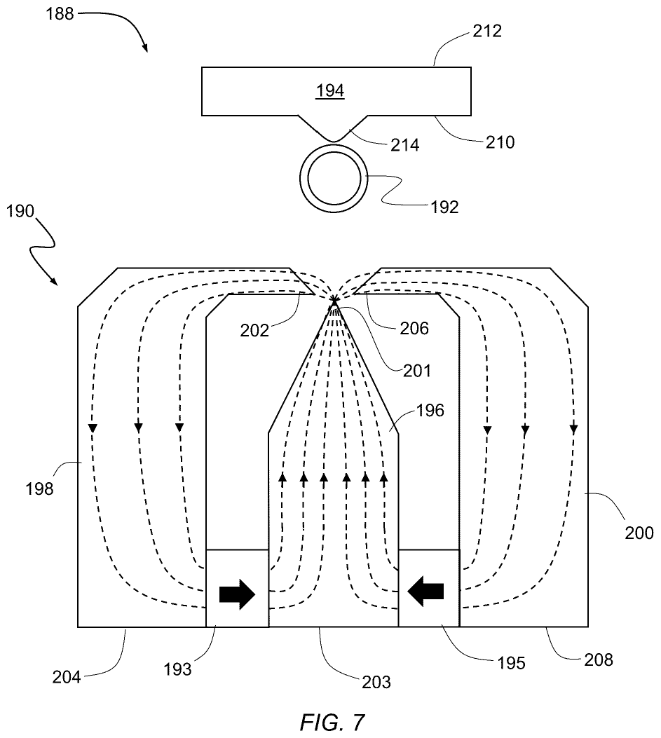

For purposes of clarity and brevity, like elements and components will bear the same designations and numbering throughout the Figures, which are not necessarily drawn to scale.

### DETAILED DESCRIPTION OF THE INVENTION

In the Summary above and in the Detailed Description, and the claims below, and in the accompanying drawings, reference is made to particular features (including method steps) of the invention. It is to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally.

Where reference is made herein to a method comprising two or more defined steps, the defined steps can be carried out in any order or simultaneously, except where the context excludes that possibility, and the method can include one or more other steps which are carried out before any of the defined steps, between two of the defined steps, or after all the defined steps, except where the context excludes that possibility.

The term “biological objects” as used herein includes cells, bacteria, viruses, molecules, particles including RNA and DNA, cell cluster, bacteria cluster, molecule cluster, and particle cluster.

The term “biological sample” as used herein includes blood, body fluid, tissue extracted from any part of the body, bone marrow, hair, nail, bone, tooth, liquid and solid from bodily discharge, or surface swab from any part of body. “Fluid sample,” or “sample fluid,” or “liquid sample,” or “sample solution” may include a biological sample in its original liquid form, biological objects being dissolved or dispersed in a buffer fluid, or a biological sample dissociated from its original non-liquid form and dispersed in a buffer fluid. A buffer fluid is a liquid into which biological objects may be dissolved or dispersed without introducing contaminants or unwanted biological objects. Biological objects and biological sample may be obtained from human or animal. Biological objects may also be obtained from plants and environment including air, water, and soil. A fluid sample may contain various types of magnetic or optical labels, or one or more chemical reagents that may be added during various process steps.

The term “sample flow rate” or “flow rate” is used herein to describe the volume amount of a fluid flowing through a cross section of a channel, a conduit, a fluidic part, a fluidic path, or a fluidic line in a unit time.

In the art of cell sorting and enrichment, the target population of biological objects is referred to as the “specific” objects of interest and those biological objects that are isolated, but are not desired, are termed “non-specific.” The term “purity” describes the frequency of target or specific biological objects of interest and is quantified by the number of target biological objects divided by the total number of biological objects expressed in percentage. The term “recovery ratio” or “recovery rate” describes the sorting efficiency of biological objects and is quantified by the number of target biological objects recovered after sorting divided by the number of target biological objects present in the initial sample expressed in percentage.

The term “at least” followed by a number is used herein to denote the start of a range beginning with that number, which may be a range having an upper limit or no upper limit, depending on the variable being defined. For example, “at least 1” means 1 or more than 1. The term “at most” followed by a number is used herein to denote the end of a range ending with that number, which may be a range having 1 or 0 as its lower limit, or a range having no lower limit, depending upon the variable being defined. For example, “at most 4” means 4 or less than 4, and “at most 40%” means 40% or less than 40%. When, in this specification, a range is given as “a first number to a second number” or “a first number-a second number,” this means a range whose lower limit is the first number and whose upper limit is the second number. For example, “25 to 100 nm” means a range whose lower limit is 25 nm and whose upper limit is 100 nm.

Directional terms, such as “front,” “back,” “top,” “bottom,” and the like, may be used with reference to the orientation of the illustrated figure. Spatially relative terms, such as “beneath,” “below,” “under,” “lower,” “upper,” “above,” etc., may be used herein to describe one element's relationship to another element(s) as illustrated in the figure. Since articles and elements can be positioned in a number of different orientations, these terms are intended for illustration purposes and in no way limit the invention, except where the context excludes that possibility.

Where reference is made herein to a material AB composed of element A and element B, the material AB can be an alloy, a compound, or a combination thereof, except where the context excludes that possibility.

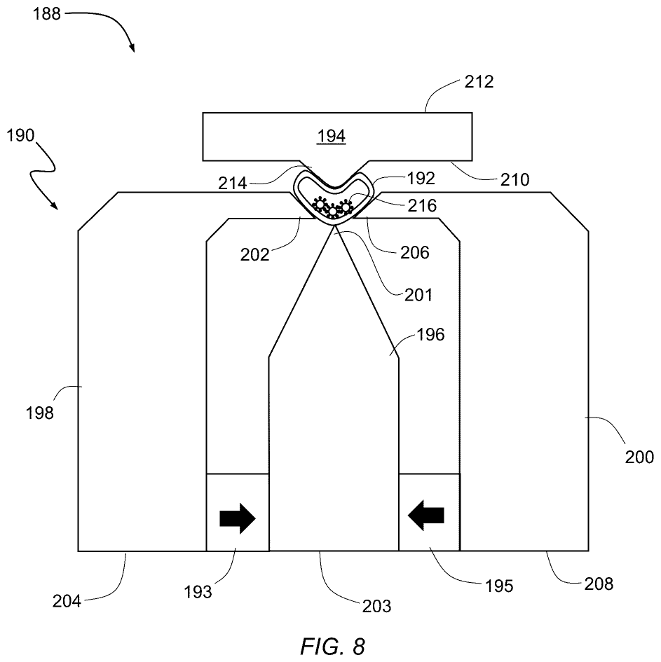

FIG. 7 is a cross-sectional view of an exemplary magnetic separator device 188 that may be used to extract and recover magnetically labeled biological objects from a suspension of heterogeneous mixture of biological objects. The magnetic separator device 188 includes a magnetic assembly 190 for generating a magnetic field, a conduit 192 made of a pliable and/or flexible material for flowing a fluid sample for sorting, and a holder 194 for supporting the conduit 192. The figure shows the conduit 192 and the holder 194 being disengaged from the magnetic assembly 190. During deposition of the magnetically labeled biological objects on the conduit wall, however, the conduit 192 would be placed in close proximity to the magnetic assembly 190, thereby exposing the conduit 192 to the magnetic field generated by the magnetic assembly 190. Unlike the column-based magnetic separator device 90, the fluid sample flowing through the conduit 192 along a direction perpendicular to the figure is unimpeded by a column of porous magnetic matrix.

The magnetic assembly 190 for generating the magnetic field to attract the magnetically labeled biological objects in the conduit 192 includes a magnetic flux source, which comprises first and second permanent magnets 193 and 195, a center magnetic flux guide 196 for conducting the magnetic flux from the magnetic flux source and forming a magnetic field, first and second side magnetic flux guides 198 and 200 disposed on opposite sides of the center magnetic flux guide 196 for conducting the magnetic flux from the magnetic flux source and forming the magnetic field at the gap between the flux guides 196-200.

The center magnetic flux guide 196 has a center tip 201 with a tapering shape and a center base 203 physically and/or magnetically coupled to the first and second permanent magnets 193 and 195 at their first pole (e.g., North pole). The center tip 201 may have a smaller cross section, which may be defined herein as the cross-sectional area perpendicular to the magnetic flux flow, than the center base 203, thereby concentrating the magnetic flux from the center base 203 to the center tip 201. The first side magnetic flux guide 198 has a first side tip 202 and a first side base 204 physically and/or magnetically coupled to the first permanent magnet 193 at its second pole (e.g., South pole). The first side tip 202 may have a smaller cross section than the first side base 204, thereby concentrating the magnetic flux from the first side base 204 to the first side tip 202. The second side magnetic flux guide 200 has a second side tip 206 and a second side base 208 physically and/or magnetically coupled to the second permanent magnet 195 at its second pole (e.g., South pole). The second side tip 206 may have a smaller cross section than the second side base 208, thereby concentrating the magnetic flux from the second side base 208 to the second side tip 206. Accordingly, each of the tips 201, 202, and 206 may have a higher magnetic flux density than the corresponding base 203, 204, or 208. The first and second side magnetic flux guides 198 and 200 may be parallel at their bases 204 and 208 and bending or kinking inward toward the center tip 201 at their tips 202 and 206, which may be pointed at each other. The ends of the first and second side tips 202 and 206 may each have a chisel edge profile with the bevel side facing upward or outward away from the center magnetic flux guide 196. The center tip 201 may be positioned below the first and second side tips 202 and 206. The conduit 192 may be operably nestled in the gap or concave space delineated by the tip end of the center tip 201 and the bevels of the first and second side tips 202 and 206 during the deposition of the magnetically labeled biological objects on the conduit wall, thereby exposing the conduit 192 to the magnetic field generated by the magnetic assembly 190.

The first permanent magnet 193 may be disposed between the center base 203 and the first side base 204, and the second permanent magnet 195 may be disposed between the center base 203 and the second side base 208. The first and second permanent magnets 193 and 195 have opposite magnetization directions that may be oriented substantially perpendicular to the center magnetic flux guide 196.

The center base 203 is magnetically coupled to the first and second permanent magnets 193 and 195 at their first pole (e.g., North pole), while the first and second side bases 204 and 208 are magnetically coupled to the first and second permanent magnets 193 and 195 at their second pole (e.g., South pole), respectively, thereby rendering the first and second side tips 202 and 206 (second polarity) and the center tip 201 (first polarity) to have opposite magnetic polarities and forming a strong magnetic field at or near the gaps between the tips 201, 202, and 206 to deposit the magnetically labeled biological objects on the conduit wall.

With continuing reference to FIG. 7, the holder 194 may have a first surface 210 facing the conduit 192 and a second surface 212 opposite the first surface 210. The first surface 210 may have a ridge structure 214 protruded from the first surface 210 that functions as a mechanical press for pushing the conduit 192 into the gap or concave space delineated by the tip end of the center tip 201 and the bevels of the first and second side tips 202 and 206 during the deposition of the magnetically labeled biological objects on the conduit wall. Additionally, the ridge structure 214 of the holder 194 may be made of a magnetic material that conducts magnetic flux like a “floating” or top magnetic flux shield. In addition to acting like a mechanical press for pushing the conduit 192 against the tips 201, 202, and 206, the ridge structure 214 made of the magnetic material may magnetically interact with the tips 201, 202, and 206 to further enhance the magnetic field therebetween, thereby increasing the magnetic sorting efficiency.

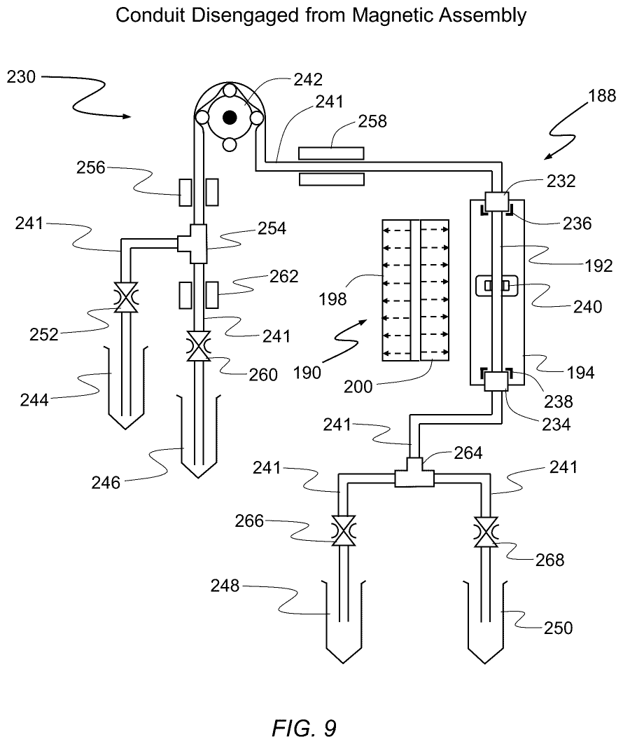

FIG. 8 is a cross-sectional view of the magnetic separator device 188 when the conduit 192 is squeezed between the ridge structure 214 of the holder 194 and the tip ends 201, 202, and 206 of the three magnetic flux guides 196-200 during the deposition of the magnetically labeled biological objects on the conduit wall. The holder 194 may push the deformed or distorted conduit 192 further into the gap between the center tip 201 and the first side tip 202 and the gap between the center tip 201 and the second side tip 206, where the magnetic field may be the strongest. Pushing the conduit 192 against the tip end of the center tip 201 and the bevels of the first and second side tips 202 and 206 may expose more fluid sample flowing through the conduit 192 to stronger magnetic field. During the deposition process, the magnetically labeled biological objects 216 may be deposited on the bottom of the conduit 192 near the center tip 201, where the magnetic field gradient may be highest.

In the embodiment where the ridge structure 214 is made of a soft magnetic material, the ridge structure 214 may act like a top magnetic flux shield when positioned in close proximity to the tips 201, 202, and 206 during the deposition of the magnetically labeled biological objects on the conduit wall. The magnetic ridge structure 214 may conduct flux from the first and second side magnetic flux guides 198 and 200 and thus may have the same magnetic polarity (second polarity) as the first and second side tips 202 and 206, thereby further enhancing the magnetic field between the ridge structure 214 and the center tip 201.

Other column-free magnetic separator devices, such as those disclosed in U.S. application Ser. No. 18/072,362, which is incorporated herein by reference, may also be employed to deposit magnetically labeled biological objects on the conduit wall during the magnetic sorting process.

The magnetic flux guides 196-200 each may be made of a soft magnetic material or a material with relatively high magnetic permeability that comprises any one of iron (Fe), cobalt (Co), nickel (Ni), or any combination thereof. For example and without limitation, any of the magnetic flux guides 196-200 may be made of iron. The conduit 192 may be made of any suitable flexible and/or pliable material that may be bent or deformed, such as but not limited to rubber, plastics, or any suitable polymeric material. The holder 194 may be made of any suitable nonmagnetic material, such as but not limited to aluminum, glass, a nonferrous metal or alloy, plastics, or any suitable polymeric material. In some embodiments, the ridge structure 214 of the holder 194 that comes into contact with the conduit 192 may be made of a soft magnetic material, such as but not limited to any of the soft magnetic materials described above for the magnetic flux guides 196-200.

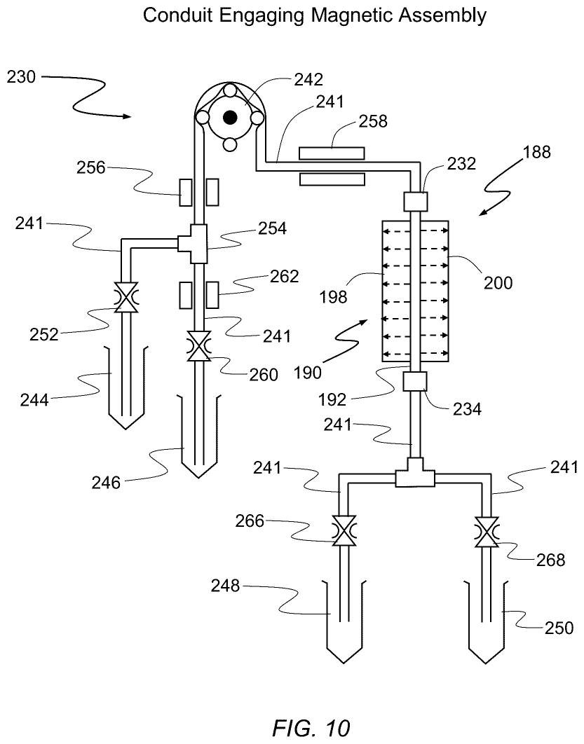

FIG. 9 is a schematic diagram depicting an exemplary fluidic circuit 230 that includes the magnetic separator device 188 when the holder 194 and conduit 192 are disengaged from the magnetic assembly 190. The conduit 192 may include two collars 232 and 234 attached thereto and may be reversibly fastened to or suspended on the holder 194 by snaping the collars 232 and 234 onto a pair of supports or brackets 236 and 238 at two ends of the holder 194. The conduit 192 may be stretched and suspended in between the brackets 236 and 238 when fastened to the holder 194, thereby ensuring that the flexible conduit 192 remains straight and aligns to the gap formed between the tips 201, 202, and 206 of the magnetic flux guides 196-200 when engaging the magnetic assembly 190 during the deposition of the magnetically labeled biological objects on the conduit wall. The holder 194 may additionally contain an opening or hole, through which an agitator arm with a fork end 240 clutching the conduit 192 may apply a transverse vibration to the conduit 192 to loosen magnetically labeled biological objects deposited on the conduit wall.

With continuing reference to FIG. 9, the conduit 192 may be a part of a network of fluidic lines 241 that uses a peristaltic pump 242 to draw fluid from a wash or buffer fluid container 244 and a fluid sample container 246 and discharge fluid to a positive collection container 248 and a negative collection container 250. Under the peristalsis effect of the peristaltic pump 242, fluid in the buffer fluid container 244 can reach the conduit 192 of the magnetic separator device 188 by passing through a first pinch valve 252, a first three-way flow connector 254, a first air detector 256, the peristaltic pump 242, and a blockage sensor 258. Similarly, fluid in the fluid sample container 246 can reach the conduit 192 of the magnetic separator device 188 by passing through a second pinch valve 260, a second air detector 262, the first three-way flow connector 254, the first air detector 256, the peristaltic pump 242, and the blockage sensor 258. Fluid discharged from the conduit 192 of the magnetic separator device 188 can be collected at the positive collection container 248, by passing through a second three-way flow connector 264 and a third pinch valve 266, and/or collected at the negative collection container 250, by passing through the second three-way flow connector 264 and a fourth pinch valve 268.

The network of fluidic lines 241 in the fluidic circuit 230 may be made of any suitable flexible and/or pliable material that may be bent or deformed, such as but not limited to rubber, plastics, or any suitable polymeric material. In an embodiment, the network of fluidic lines 241 and the conduit 192 are made of the same flexible and/or pliable material. The network of fluidic lines 241, including the conduit 192, may be constructed, interconnected, and supplied as a disposable tubing set. Additional connectors or different types of connectors or fittings may also be used to construct the network of fluidic lines 241 in the fluidic circuit 230.

The first and second pinch valves 252 and 260 may be used to regulate the flow of fluid from/to the buffer fluid container 244 and the fluid sample container 246, respectively. The third and fourth pinch valves 266 and 268 may be used to regulate the flow of fluid to the positive and negative collection containers 248 and 250, respectively. The pinch valves 252, 260, 266, and 268 close the flow of fluid in the fluidic lines 241 by squeezing the walls of a flexible fluidic line against one another. The first and second air detectors 256 and 262, which use ultrasound to sense air bubbles or air gaps in the fluidic lines 241 before the peristaltic pump 242, may detect leaks in the fluidic lines 241 or depletion of the buffer fluid and fluid sample in the buffer fluid container 244 and the fluid sample container 246, respectively. The blockage sensor 258, which is disposed along the fluidic line between the peristaltic pump 242 and the magnetic separator device 188, uses capacitive sensing to detect the blockage of flow through the magnetic separator device 188 and beyond. The operation of the electromechanical components 242, 252, 256-262, 266, and 268 may be controlled by a central process unit (CPU) or computer (not shown).

In the embodiment shown in FIG. 9, the peristaltic pump 242, the pinch valves 252, 260, 266, and 268, the air sensors 256 and 262, and the blockage sensor 258 are external to the flexible fluidic lines 241 of the fluidic circuit 230, thereby preventing direct contact between the fluid in the fluidic lines 241 and these components 242, 252, 256-262, 266, and 268. However, it should be understood that the fluidic circuit 230 may alternatively be constructed using other types of pumps, sensors, valves, or fluidic lines. For example, a portion of the network of fluidic lines 241 may be made of a rigid material, which may require the use of other types of pump and valves that are positioned in the fluid pathways and thus come into direct contact with the fluid flowing through the rigid fluidic lines.

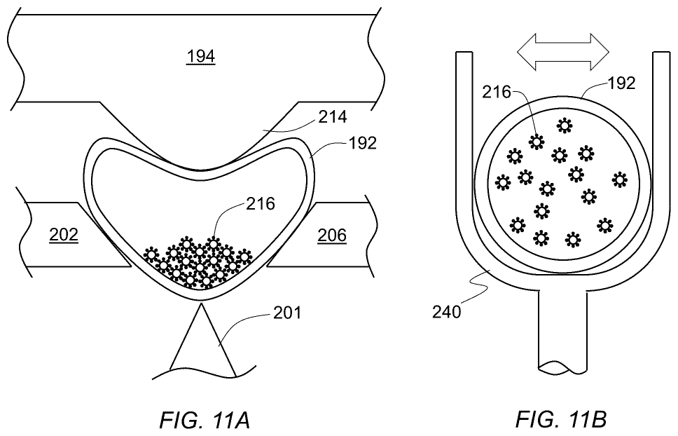

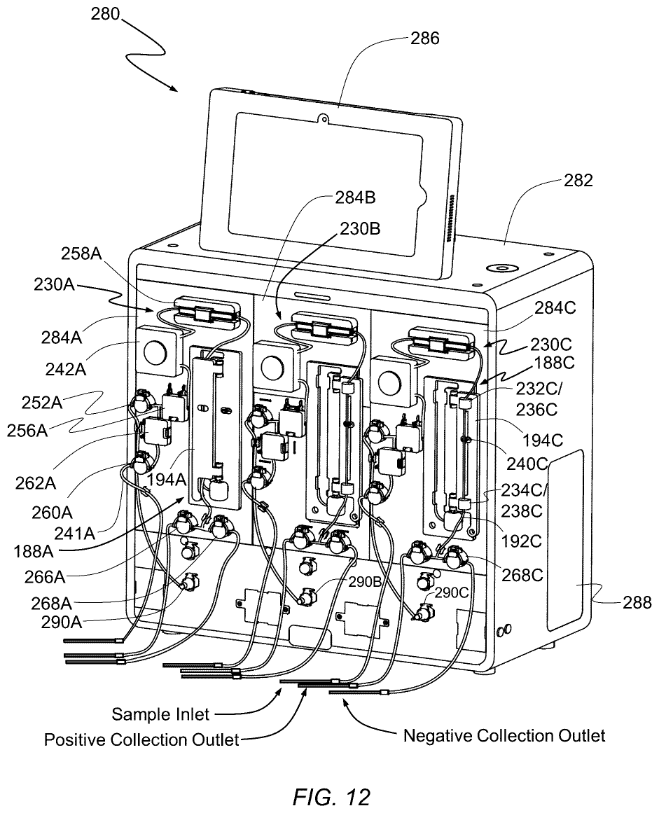

Operation of the fluidic circuit 230 for magnetic sorting will now be described with reference to the schematic diagram of FIG. 10, which shows the conduit 192 engaging the magnetic assembly 190. The holder 194, which presses the conduit 192 against the magnetic assembly 190 during the deposition of the magnetically labeled biological objects on the conduit wall (as shown in FIG. 8), is omitted in FIG. 10 for reasons of clarity. An exemplary process of magnetic sorting begins by providing a fluid sample including a mixture of magnetically labeled and unlabeled biological objects in the fluid sample container 246. The fluid sample is pumped by the peristaltic pump 242 into the conduit 192 engaging the magnetic assembly 190, with the first and third pinch valves 252 and 266 shut and the second and fourth pinch valves 260 and 268 opened. As the fluid sample flows through the conduit 192 that is exposed to the magnetic field generated by the magnetic assembly 190, the magnetically labeled biological objects 216 are attracted by the magnetic field and are deposited or collected on the conduit wall as shown in FIG. 11A, while the nonmagnetic or unlabeled biological objects in the depleted fluid sample are flushed into the negative collection container 250 with the depleted fluid sample.

After the passage of the fluid sample through the conduit 192, a rinse process may be used to further remove debris or unlabeled biological objects from the deposited magnetically labeled biological objects on the conduit wall by flowing a buffer fluid through the conduit 192 while the conduit 192 remains engaged to the magnetic assembly 190. The flow between the sample fluid for deposition and the buffer fluid for rinse may be continuous without any air gap therebetween. After the optional rinse process, the conduit 192 may be filled with the buffer fluid in the stagnant state.

After the separation of the magnetically labeled biological objects deposited on the conduit wall of the magnetic separator device 188 from the unlabeled biological objects in the negative collection container 250 is completed, the process of recovering the magnetically labeled biological objects proceeds by moving the holder 194 and the conduit 192 away from the magnetic assembly 190 and the magnetic field generated thereby, as shown in FIG. 9. However, simply removing the conduit 192 from the magnetic field and flushing the conduit 192 with buffer fluid may not cause the accumulation or buildup of magnetically labeled biological objects on the conduit wall to dislodge from the conduit wall and/or dissociate into individual biological objects for recovery, because the magnetic beads on a biological object may still experience magneto-static field from neighboring magnetic beads and/or magnetic beads of neighboring biological objects. Therefore, mechanical agitation may be applied to the conduit 192 by the agitator arm 240, which has one end attached to a vibration source and another end (i.e., fork end) clutching the conduit 192, to loosen the magnetically labeled biological objects 216 deposited on the conduit wall in the presence of a static or moving buffer fluid as shown in FIG. 11B.

With continuing reference to FIG. 9, during the recovery process, a buffer fluid in the buffer fluid container 244 is pumped into the conduit 192 by the peristaltic pump 242, with the second and fourth pinch valves 260 and 268 shut and the first and third pinch valves 252 and 266 opened. As the buffer fluid flows through the conduit 192, the agitator arm 240 may impart transverse vibration to the conduit 192 to loosen and dissociate the accumulation or buildup of magnetically labeled biological objects on the conduit wall. The buffer fluid carrying the magnetically labeled biological objects then flows from the conduit 192 into the positive collection container 248. Alternatively, the recovery of the magnetically labeled biological objects from the conduit wall may be carried out by imparting the transverse vibration to the conduit 192 while the flow of the buffer fluid is temporarily halted when the conduit 192 is filled with the buffer fluid, thereby resuspending the magnetically labeled biological objects in the buffer fluid contained in the conduit 192. The application of the transverse vibration under the static fluid condition may reduce the amount of buffer fluid required for the recovery process. After the elution of the magnetically labeled biological objects deposited on the conduit wall with the buffer fluid, additional buffer fluid may be added to dilute the fluid sample in the positive collection container 248 and resuspend the magnetically labeled biological objects for subsequent process. Alternatively, the elution of the magnetically labeled biological objects may be carried out by using excess buffer fluid to dilute the fluid sample without the separate dilution step.

The operation of the fluidic circuit 230 for magnetic sorting as described above may be automated by a computer program executed by a CPU or computer without manual intervention by the operator.

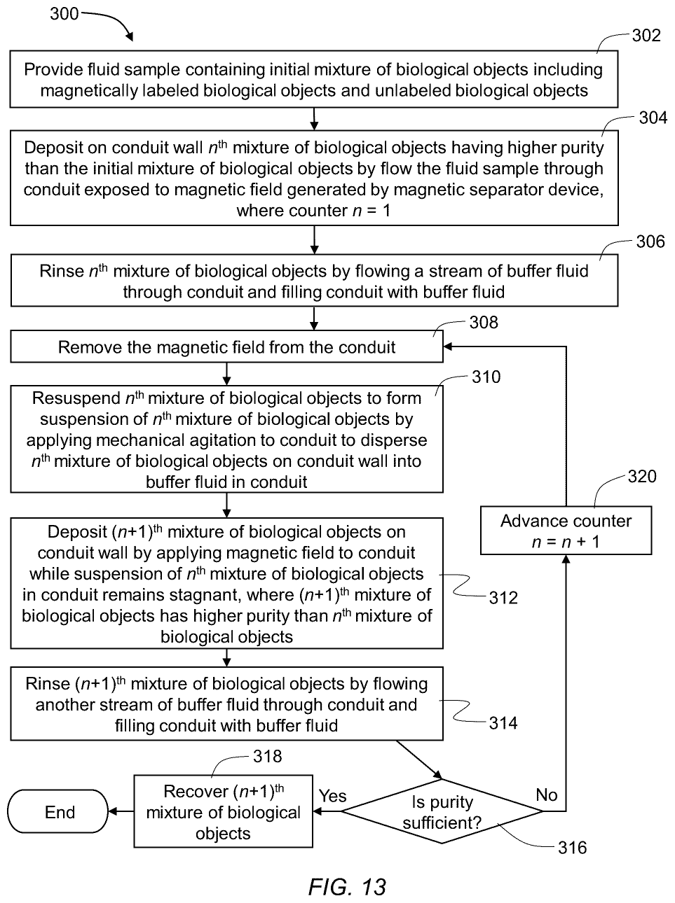

The fluidic circuit 230 including the magnetic separator device 188, as shown in FIGS. 9 and 10, may be implemented as part of a magnetic separator system 280 shown in a perspective view in FIG. 12. The magnetic separator system 280 includes a system housing 282, first, second, third magnetic separator modules 284A-284C residing in the housing 282, and an external computer 286 for controlling the electrical and electromechanical components in the system 280. The three magnetic separator modules 284A-284C may be substantially identical. A rack for holding the containers 246-250, which is normally disposed in front of the system housing 282 and the modules 284A-284C, and the containers 246-250 are omitted in the drawing in order to present an unobstructive view of the modules 284A-284C.

Each of the first, second, and third magnetic separator modules 284A-284C includes a corresponding fluidic circuit 230A, 230B, or 230C. In the configuration shown, all three fluidic circuits 230A-230C draw a wash or buffer fluid from a common container or tank (not shown) disposed inside the system housing 282. The common container can be access through a side panel door 288 and the wash or buffer fluid stored in the common container can be extracted through three inlet ports 290A-290C disposed on the front of the system housing 282.

Each of the first, second, and third fluidic circuits 230A-230C includes a network of fluidic lines and fluidic components analogous to those of the fluidic circuit 230 shown in FIGS. 9 and 10. For example and without limitation, the first fluidic circuit 230A includes a network of fluidic lines 241A, first and second pinch valves 252A and 260A for regulating the flow of fluid from/to the first inlet port 290A and the sample inlet, respectively; and third and fourth pinch valves 266A and 268A for regulating the flow of fluid to the positive and negative collection outlets, respectively; a magnetic separator device 188A; a peristaltic pump 242A for moving fluid from the inlets of the network of fluidic lines 241A, through the magnetic separator device 188A, to the collection outlets of the network of fluidic lines 241A; first and second air detectors 256A and 262A for sensing air bubbles or air gaps in the fluidic lines 241A before the peristaltic pump 242A; and a blockage sensor 258A disposed between the peristaltic pump 242A and the magnetic separator device 188A for detecting the blockage of flow through the magnetic separator device 188A and beyond. The second and third fluidic circuits 230B and 230C are substantially identical to the first fluidic circuit 230A and have the same fluidic components. However, not all fluidic components of the second and third fluidic circuits 230B and 230C are explicitly labeled in FIG. 12 for reasons of legibility of the drawing.

Each of the magnetic separator modules 284A-284C may further include various electrical components (not shown) connected to assorted electrical components and a power supply in the system housing 282. The computer 286, which is connected to the system housing 282, may control the electrical and electromechanical components in the system 280 to allow individual magnetic separator modules 284A-284C to operate independently. For example and without limitation, in the configuration shown in FIG. 12, the holder 194A of the first magnetic separator module 284A clamps the flexible conduit against the magnetic assembly (analogous to FIG. 8), while the holder 194C of the third magnetic separator module 284C and the conduit 192C move away from the magnetic assembly with the conduit 192C engaging the agitator arm 240C through an opening or hole in the holder 194C (analogous to FIG. 9). The entire magnetic sorting process may also be automated by the computer 286 without manual intervention by the operator.

The standard magnetic sorting process involving deposition, rinse, and recovery as described above may attain high purity and recovery ratio if the initial target cell frequency or purity of the fluid sample is not too low (e.g., above 1%). For magnetically labeled biological objects that only have a miniscule presence in the fluid sample (e.g., less than 1%), the unlabeled biological objects may still account for several tens of percent of all biological objects deposited on the conduit wall even after removing a great majority (e.g., greater than 99%) of the unlabeled biological objects with the depleted fluid sample. Therefore, the above-described magnetic sorting process may have to be repeated one or more times to attain sufficiently high target biological object purity. However, doing so may adversely decrease the recovery ratio if the magnetic sorting process is not properly optimized.

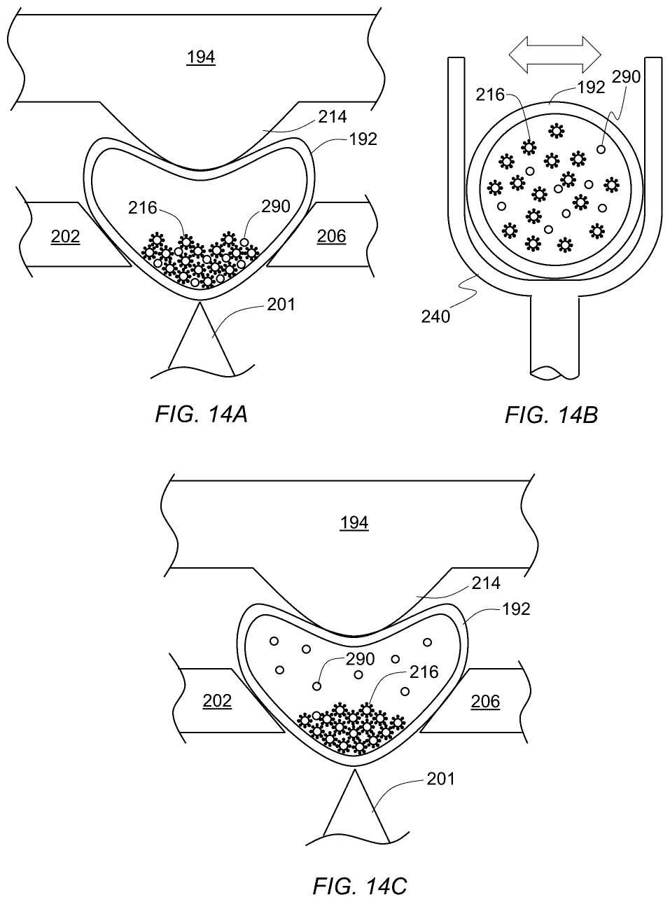

The present invention aims to increase the frequency of the magnetically labeled biological objects or magnetically labeled biological object purity while substantially maintaining the recovery ratio during magnetic sorting, especially for fluid samples that contain only miniscule amount of target biological objects. FIG. 13 is a flow chart illustrating selected steps 300 for magnetically sorting biological objects in accordance with an embodiment of the present invention. The process steps 300 begin at step 302 by providing a fluid sample containing an initial mixture of biological objects including magnetically labeled biological objects and unlabeled biological objects. The magnetically labeled biological objects may be cells that express target antigen with magnetic beads directly or indirectly attached thereto. The unlabeled biological objects do not have magnetic beads attached thereto and may include debris or cells that do not express target antigen.

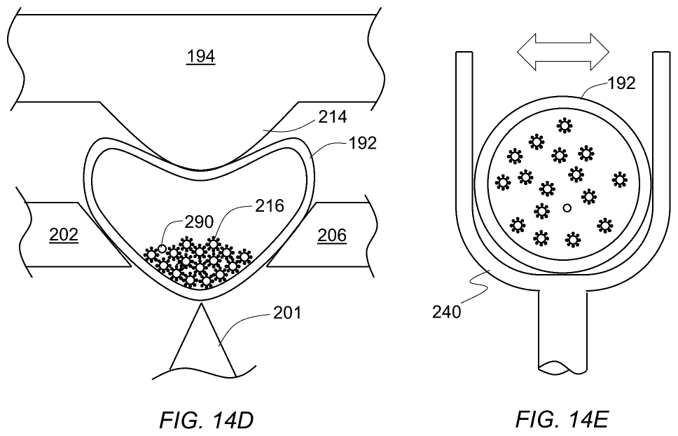

Next, at step 304, a first (n^{th}, n=1) mixture of biological objects, which has a frequency of the magnetically labeled biological objects or magnetically labeled biological object purity higher than the initial mixture of biological objects, is deposited on a conduit wall of a magnetic separator device by flowing the fluid sample through a conduit exposed to a magnetic field generated by a magnetic separator device. This process step may be carried out using the fluidic circuit 230 of FIG. 10. The fluid sample may flow from the fluid sample container 246 to the magnetic separator device 188 and the depleted fluid sample may flow from the magnetic separator device 188 to the negative collection container 250. Any column-free continuous flow magnetic separator device that can generate sufficiently high magnetic field and field gradient, such as but not limited to the magnetic separator device 188 shown and described above, may be used to generate the magnetic field to attract the magnetically labeled biological objects to the conduit wall. Using the magnetic separator device 188 (FIGS. 8, 10) as an example, the fluid sample is flowed through the conduit 192 to deposit the first mixture of biological objects on the conduit wall, which include magnetically labeled biological objects 216 and unlabeled biological objects 290 as shown in FIG. 14A. While a great majority portion of the unlabeled biological objects exits the magnetic separator device 188 with the depleted fluid sample, the first mixture of biological objects on the conduit wall may still contain appreciable number of unlabeled biological objects, albeit significantly less than the initial mixture of biological objects. Accordingly, the first mixture of biological objects has a higher frequency of the magnetically labeled biological objects or magnetically labeled biological object purity than the initial mixture of biological objects.

After the fluid sample flows through the conduit, the first (n^{th}, n=1) mixture of biological objects deposited on the conduit wall may be optionally rinsed by flowing a stream of buffer fluid through the conduit to further remove debris or unlabeled biological objects from the first mixture of biological objects and filling the conduit with the buffer fluid at step 306 while the conduit remains exposed to the magnetic field generated by the magnetic separator device. The flow between the sample fluid for deposition and the buffer fluid for rinse may be continuous without any air gap therebetween. After the optional rinse process, the first mixture of biological objects on the conduit wall may be immersed in the stagnant or static buffer fluid. When using the fluidic circuit 230 of FIG. 10, as the last of the fluid sample is drawn out of the fluid sample container 246, the second pinch valve 260 closes and the first pinch valve 252 opens to draw the buffer fluid behind the fluid sample. The fluid sample and the buffer fluid may flow in a contiguous manner without any air gap therebetween. After the last of the depleted fluid sample and rinsed buffer fluid flow through the fourth pinch valve 268 and into the negative collection container 250, the valve 268 is closed, thereby trapping a volume of the buffer fluid in the conduit 192 spanning the magnetic separator device 188 to immerse the first mixture of biological objects in the buffer fluid.

After the deposition and optional rinse at steps 304 and 306, the magnetic field is removed from the conduit and the first mixture of biological objects deposited therein at step 308. If the optional rinse process at step 306 was not used to fill the conduit, the conduit spanning the magnetic separator device may be filled with the buffer fluid after the removal of the magnetic field, thereby immersing the first mixture of biological objects in the buffer fluid. When using the fluidic circuit 230 of FIG. 10, step 308 may be practiced by disengaging the conduit 192 from the magnetic assembly 190 and moving the holder 194 and the conduit 192 to the recovery position shown in FIG. 9. For some magnetic separator devices, such as the magnetic separator device 188 shown in FIG. 8, the interior volume of the conduit 192 is squeezed when engaging the magnetic assembly 190 during the deposition step 304. The disengagement of the conduit 192 from the magnetic assembly 190 will cause the internal volume of the conduit 192 to expand, which may result in insufficient buffer fluid to cover all of the first mixture of biological objects on the conduit wall. Accordingly, additional buffer fluid may be added to the conduit 192 such that the portion of the conduit 192 spanning the magnetic separator device 188 is filled with the buffer fluid.

After the removal of the magnetic field from the conduit and immersion of the first mixture of biological objects deposited on the conduit wall in the stagnant or static buffer fluid, the first (n^{th}, n=1) mixture of biological objects is resuspended in the buffer fluid to form a suspension of the first mixture of biological objects by applying mechanical agitation to the conduit to disperse the first mixture of biological objects deposited on the conduit wall into the stagnant buffer fluid in the conduit at step 310. This step may be practiced using the fluidic circuit 230 at the recovery position shown in FIG. 9, with the fork end of the agitator arm 240 clutching the conduit 192. The agitator arm 240 may exert transverse vibration on the conduit 192 to disperse the first mixture of biological objects, which includes magnetically labeled biological objects and unlabeled biological objects, from the conduit wall into the buffer fluid to form the suspension of the first mixture of biological objects, as shown in FIG. 14B. For magnetic separator devices that use a rigid conduit, mechanical agitation in the form of ultrasonic vibration may be applied to the rigid conduit by a piezoelectric transducer to disperse the first mixture of biological objects into the buffer fluid.

Referring back to FIG. 13, the process continues by depositing on the conduit wall a second ((n+1)^{th}, n=1) mixture of biological objects, which has another frequency of the magnetically labeled biological objects or magnetically labeled biological object purity higher than the first (n^{th}, n=1) mixture of biological objects, by applying the magnetic field again to the conduit at step 312 while the suspension of the first mixture of biological objects in the conduit remains stagnant. This static magnetic deposition step 312 may be practiced by reengaging the conduit 192 of the fluidic circuit 230 to the magnetic assembly 190 (FIG. 10), which applies the magnetic field to the conduit 192 to deposit the second mixture of biological objects as shown in FIG. 14C. In the previous deposition of the first mixture of biological objects on the conduit wall as the fluid sample flows through the conduit 192 at step 304, the magnetically labeled biological objects 216 in the fluid sample may only have a limited time for deposition ranging from several seconds to several tens of seconds, depending on the flow rate of the fluid sample as it passes through the magnetic separator device 188. By contrast, the deposition of the second mixture of biological objects on the conduit wall occurs while the suspension of the first mixture of biological objects in the conduit remains essentially stagnant or static. The lack of hydrodynamic force to hinder the deposition of the magnetically labeled biological objects and/or the potential increase in deposition time at the static deposition step 312 may provide favorable conditions for recovering most, if not all, of the magnetically labeled biological objects 216 previously recovered as part of the first mixture of biological objects. At the same time, most of the unlabeled biological objects 290 previously recovered as part of the first mixture of biological objects would remain suspended in the depleted suspension of the first mixture of biological objects. Therefore, the second mixture of biological objects deposited on the conduit wall has another frequency of the magnetically labeled biological objects or magnetically labeled biological object purity higher than the first mixture of biological objects.

It is worth noting that when using the fluidic circuit 230 shown in FIG. 10 as an example to practice the static deposition step 312, the suspension of the first mixture of biological objects is contained in the conduit 192 by shutting the third and fourth pinch valves 266 and 268. Therefore, there may exist a small portion of the suspension of the first mixture of biological objects between the magnetic separator device 188 and the valves 266 and 268 that is not exposed to the magnetic field. Such portion of the suspension may be moved into the magnetic separator device 188 by reversely pumping the suspension to perform the static deposition.

After the static deposition at step 312, the second ((n+1)^{th}, n=1) mixture of biological objects deposited on the conduit wall may be rinsed by flowing another stream of fresh buffer fluid through the conduit to remove the unlabeled biological objects suspended in the depleted buffer fluid in the conduit and to further remove debris or unlabeled biological objects from the second mixture of biological objects deposited on the conduit wall at step 314 while the conduit remains exposed to the magnetic field generated by the magnetic separator device. The conduit may be filled with fresh buffer fluid to immerse the second mixture of biological objects in the fresh buffer fluid at the end of step 314. This rinse step 314 is analogous to the optional rinse step 306 described above and may be practiced using the fluidic circuit 230 shown in FIG. 10.

After the rinse step 314, the process continues to step 316, where a decision is made as to whether the frequency of magnetically labeled biological objects or magnetically labeled biological object purity of the second ((n+1)^{th}, n=1) mixture of biological objects is sufficiently high enough. If so, the process advances to step 318 to recover the second ((n+1)^{th}, n=1) mixture of biological objects from the conduit wall.

The recovery of the second ((n+1)^{th}, n=1) mixture of biological objects deposited on the conduit wall at step 318 begins by removing the magnetic field from the conduit and then flowing still another stream of fresh buffer fluid through the conduit to elute the second mixture of biological objects. The conduit may be subjected to mechanical agitation prior to or during the elution process to help loosen the second mixture of biological objects from the conduit wall. The recovery step 318 may be practiced with the fluidic circuit 230 by disengaging the conduit 192 from the magnetic assembly 190 and moving the holder 194 and the conduit 192 to the recovery position shown in FIG. 9, with the fork end of the agitator arm 240 clutching the conduit 192. The agitator arm 240 may exert transverse vibration on the conduit 192 to disperse the second mixture of biological objects into the buffer fluid in the conduit 192 prior to or during the elution process. The eluted second mixture of biological objects, along with the buffer fluid, is then recovered at the positive collection container 248. The use of the hydrodynamic force of flowing buffer fluid in combination mechanical vibration may further enhance the recovery process of the second mixture of biological objects from the conduit wall. The magnetic sorting process ends after the recovery of the second mixture of biological objects step 318.

If the frequency of magnetically labeled biological objects or magnetically labeled biological object purity of the second mixture of biological objects is not sufficiently high enough at step 316, then the loop counter n is incremented by 1 (i.e., n=2) at step 320, and the static magnetic sorting of steps 308-314 are repeated to deposit a third ((n+1)^{th}, n=2) mixture of biological objects, which has still another frequency of the magnetically labeled biological objects or magnetically labeled biological object purity higher than the second (n^{th}, n=2) mixture of biological objects, on the conduit wall of the conduit filled with a suspension of the second mixture of biological objects in a static or stagnant state. Steps 308-314 may be practiced using the fluidic circuit 230 shown in FIGS. 9 and 10 as described above.

At step 316, where a decision is made as to whether the frequency of magnetically labeled biological objects or magnetically labeled biological object purity of the third ((n+1)^{th}, n=2) mixture of biological objects is sufficiently high enough. If so, the process advances to step 318 to recover the third ((n+1)^{th}, n=2) mixture of biological objects as described above and ends thereafter. Otherwise, the loop counter n is further incremented by 1 (i.e., n=3) at step 320, and steps 308-314 are repeated to deposit a fourth ((n+1)^{th}, n=3) mixture of biological objects, which has yet another frequency of the magnetically labeled biological objects or magnetically labeled biological object purity higher than the third (n^{th}, n=3) mixture of biological objects, on the conduit wall of the conduit filled with a suspension of the third mixture of biological objects in a static or stagnant state. Steps 308-314 for static magnetic sorting may be repeated as many times as needed until desire purity is achieved.

The preparation process for the initial fluid sample for magnetic sorting, which contains an initial mixture of magnetically labeled biological objects and unlabeled biological objects, may begin by adding a magnetic reagent to an initial raw sample (e.g., whole blood) for directly labeling the biological objects with magnetic beads. Alternatively, the target biological objects may be indirectly labeled by first adding a reagent containing intermediate links, such as phycoerythrin (PE) conjugated with the targeted antibody, to the initial raw sample, thereby attaching the intermediate links to the target biological objects after an incubation period. A magnetic reagent containing magnetic beads that target the intermediate links may then be added to the mixture of initial raw sample and the reagent containing intermediate links to complete the indirect magnetic labeling process after another incubation period. A buffer fluid may be added to the initial fluid sample containing the target biological objects before or after the magnetic labeling process to adjust the concentration of the biological objects in the initial fluid sample and/or the viscosity of the initial fluid sample. The dilution of the initial raw sample with the buffer fluid may reduce coalescence and clumping of biological objects and/or reduce potential obstruction by unlabeled biological objects as magnetically labeled biological objects move toward conduit wall under the influence of the external magnetic field exerted by the magnetic separator device, thereby facilitating the sorting process.

### EXAMPLES

The following examples are provided to illustrate, but not limit the invention. The recovery rate of target biological objects reported herein is calculated from the number of events or cells for the magnetically labeled target biological objects in the fluid sample after magnetic sorting divided by the number of events or cells for the magnetically labeled target biological objects in the initial fluid sample prior to any sorting, as measured by a flow cytometer (CytoFlex, Beckman Coulter). The frequency of the magnetically labeled biological objects or magnetically labeled biological object purity reported herein is calculated from the number of events or cells for the magnetically labeled target biological objects divided by the number of all events or biological objects in the fluid sample, as measured by the flow cytometer. The same buffer fluid (MARS® MAG Buffer, Applied Cells) is used during sample preparation and magnetic sorting process.

Each example described herein is carried out using one of the three modules 284A-284C of the magnetic separator system 280 shown in FIG. 12 to automate the processing steps, unless noted otherwise. After the initial fluid sample is added to the fluid sample container of one of the modules (e.g., 284C), no manual intervention is needed until the final fluid sample exits the positive collection outlet of the module 284C.

The sequential magnetic sorting steps begin by adding an initial volume of the initial fluid sample, which contains an initial mixture of magnetically labeled biological objects and unlabeled objects, to the fluid sample container for the module 284C of the magnetic separator system 280. After drawn through the sample inlet, the initial fluid sample passes through the conduit 192C of the magnetic separator device 188C of the module 284C at a first flow rate to deposit on the conduit wall the first mixture of biological objects, which has a higher magnetically label biological object purity than the initial mixture. After deposition, the first mixture of biological objects on the conduit wall is rinsed with the buffer fluid and then immersed in the buffer fluid. After rinsing, the holder 194C moves the conduit 192C filled with the buffer fluid away from the magnetic assembly 190C to the recovery position (i.e., FIG. 9) with the agitator arm 240C clutching the conduit 192C. An additional amount (˜0.1 mL) of buffer fluid is added to the conduit 192C to fill the conduit 192 spanning the magnetic separator device 188C because of the interior volume expansion of the conduit 192C, which causes the buffer fluid level to drop, after disengagement from the magnetic assembly 190C. Transverse vibration is then applied to the conduit 192C through the agitator arm 240C for about 24 s to resuspend the first mixture of biological objects in the buffer fluid in the conduit 192C to form a suspension of the first mixture of biological objects. Afterward, the holder 194C moves the conduit 192C back to reengage the magnetic assembly (i.e., FIG. 10) for static magnetic deposition for about 40 s to deposit on the conduit wall a second mixture of biological objects, which has a higher magnetically labeled biological object purity than the first mixture. The suspension of the first mixture of biological objects in the conduit 192C remains stagnant or static during this static magnetic deposition step. After the static magnetic deposition step, the second mixture of biological objects on the conduit wall is rinsed with a fresh buffer fluid to remove the depleted buffer fluid containing the unlabeled biological objects from the conduit 192C and to further remove debris and unlabeled biological objects from the second mixture of biological objects while the conduit 192C remains engaged to the magnetic assembly 192C. After the rinse process, the conduit 192C is filled with the fresh buffer fluid.

The static magnetic sorting steps described above may be repeated to deposit a third mixture of biological objects, which has a higher magnetically label biological object purity than the second mixture of biological objects. The static magnetic sorting steps may be repeated as many times as needed to attain a final mixture of biological objects that has the desired purity.

The recovery of the second or final mixture of biological objects with desired purity proceeds by moving the holder 194C and the conduit 192C filled with the buffer fluid to the recovery position (i.e., FIG. 9) with the agitator arm 240C clutching the conduit 192C. The second or final mixture of biological objects deposited on the conduit wall is eluted with about 1.2 mL of the buffer fluid while the agitator arm 240C applies transverse vibration to the conduit 192C.

For reference purpose, an aliquot of the initial fluid sample, which contains the initial mixture of magnetically labeled biological objects and unlabeled objects, is also magnetically sorted using the standard sorting process that includes deposition of the first mixture of biological objects on the conduit wall by passing the aliquot of the initial fluid sample through the conduit 192C, subsequent rinse step, and recovery of the first mixture of biological objects without additional static magnetic sorting steps (i.e., single pass only).

### Example 1: Enrichment of CD34+ Hematopoietic Stem Cells (HSCs) in Mononuclear Cells (MNCs) Derived from Human Cord Blood

Hematopoietic stem cells (HSCs), which can produce all the other cells found in blood and treat various blood diseases, are present in human umbilical cord blood. HSCs have CD34 antigen expression and therefore can be magnetically labeled and sorted accordingly.

The sample preparation process begins by extracting the buffy coat from a human cord blood sample using centrifugation over a density gradient medium (Ficoll Paque™ Plus, Cytiva). A mononuclear cell (MNC) suspension is then prepared from the buffy coat by further centrifugation over the density gradient medium. The MNC sample (i.e., initial raw sample) is resuspended in a buffer fluid (MARS® MAG Buffer, Applied Cells) at a concentration of about 10⁸ cells/0.3 mL of buffer fluid. The target cells (i.e., HSCs) in the resuspended MNC sample are magnetically labeled by adding a reagent containing FcR blocker (FcR Blocking Reagent, human, Miltenyi Biotec) at a dosage of 0.1 mL per 10⁸ cells for minimizing unspecific binding and a reagent containing magnetic beads (CD34 MicroBeads, human, ˜50 nm in diameter, Miltenyi Biotec) at a dosage of 0.1 mL per 10⁸ cells to the resuspended MNC sample. The mixture is incubated for 30 min at 4° C. while rocking on a mixer (Nutating Mixer, Labnet International). The magnetically labeled MNC sample is further diluted with about 3 times the volume of buffer fluid (i.e., 4-fold dilution) and gently mixed by pipetting up and down to yield the initial fluid sample for magnetic sorting.

An aliquot of the initial fluid sample is flowed through the magnetic separator system 280 at a flow rate of 0.2 mL/min to deposit a first mixture of biological objects on the conduit wall, after which the first mixture of biological objects is further sorted using the static magnetic sorting process described above, resulting in the second mixture of biological objects in the final sample. Another aliquot of the initial fluid sample is flowed through the magnetic separator system 280 at the same flow rate of 0.2 mL/min to obtain the standard sample containing the first mixture of biological objects without further static magnetic sorting (i.e., one-pass-only sorting).

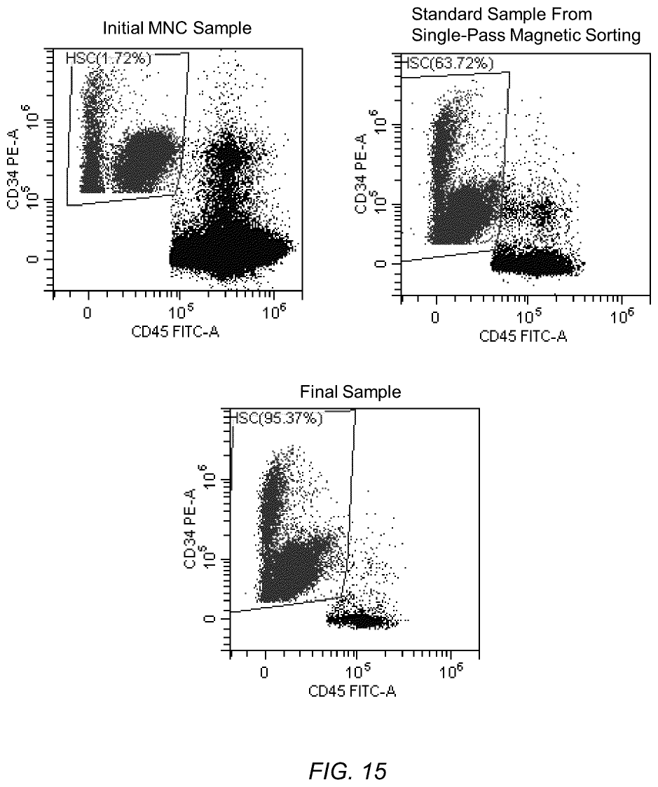

The compositions of the initial MNC, standard, and final samples, as measured by the flow cytometer (CytoFlex, Beckman Coulter) are shown in the dot plots of FIG. 15 and summarized in Table 1 below. Prior to cytometry measurement, an aliquot of each of the initial, standard, and final samples is stained with fluorescently conjugated antibodies: CD34 PE for stem cells and CD45 FITC for mononuclear cells. The cytometry results show the final sample sorted in accordance with an embodiment of the present invention has a relatively high recovery ratio of 78.48% and 95.37% stem cell purity compared with 1.72% stem cell purity of the initial MNC sample. Compared with the standard process (i.e., single-pass-only sorting), which produces the standard sample, the current invention increases the stem cell purity from 63.72% to 95.37% with only a slight decrease in the recovery ratio.

TABLE 1
Stem cell purity and recovery ratio for initial MNC, standard, and
final samples

| Sample | Purity | Recovery ¹ |
|---|---|---|
| Initial MNC sample | 1.72% | |
| Standard sample (single-pass-only sorting) | 63.72% | 84.24% |
| Final sample (with additional static sorting) | 95.37% | 78.48% |

¹ Compared with initial MNC sample

### Example 2: Enrichment of Multiple Myeloma Cells in in Peripheral Blood Mononuclear Cells (PBMCs) Spiked with CD138+ MM1S Cells

Multiple myeloma is characterized by the expansion of malignant plasma cells within the bone marrow. While these malignant plasma cells can be readily identified by their high expression of CD38 and B-B4, their purification and in vitro expansion remain difficult. Therefore, established human myeloma cell lines, such as MM1S, are commonly used to study the biology of multiple myeloma. In this example, a peripheral blood mononuclear cell (PBMC) sample is spiked with MM1S cells to simulate the blood of a multiple myeloma patient.

The sample preparation process begins by extracting the buffy coat from a peripheral blood sample using centrifugation over a density gradient medium (Ficoll Paque™ Plus, Cytiva). A PBMC suspension is then prepared from the buffy coat by further centrifugation over the density gradient medium. The PBMCs extracted from the buffy coat is resuspended in a buffer fluid (MARS® MAG Buffer, Applied Cells) at a concentration of about 10⁸ cells/0.3 mL of buffer fluid. MM1S cells pre-stained with fluorescently conjugated antibodies CD298 APC (APC anti-human CD298 Antibody, Biolegend) are then added to the PBMC sample and gently mixed by pipetting up and down.

The target cells (i.e., MM1S cells) in the MM1S spiked PBMC sample are magnetically labeled in an indirect process by first adding a first reagent containing intermediate links directed to CD138 antigen expression (EasySep™ Human CD138 Positive Selection Cocktail, STEMCELL Technologies) to the MM1S spiked PBMC sample at a ratio of about 50 μL reagent per mL of PBMC sample. The mixture is incubated for 8 min at room temperature while rocking on a mixer (Nutating Mixer, Labnet International), allowing time for the primary antibodies of the intermediate links to attach to the CD138 antigens on the target cell surface. A second reagent containing magnetic beads (EasySep™ Dextran RapidSpheres™ 50100, ˜150 nm in diameter, STEMCELL Technologies) is then added to the previously incubated mixture at a ratio of about 50 μL reagent per mL of PBMC sample to attach the magnetic beads to the intermediate links. After incorporating the second reagent containing the magnetic beads, the PBMC sample is allowed to incubate for 8 min at room temperature while rocking on the mixer to complete the magnetic labeling process. After the magnetic labeling process, the PBMC sample is further diluted with about 1 time the volume of buffer fluid (i.e., 2-fold dilution) and gently mixed by pipetting up and down to yield the initial fluid sample for magnetic sorting.

An aliquot of the initial fluid sample is flowed through the magnetic separator system 280 at a flow rate of 0.8 mL/min to deposit a first mixture of biological objects on the conduit wall, after which the first mixture of biological objects is further sorted using the static magnetic sorting process described above, resulting in the second mixture of biological objects in the final sample. Another aliquot of the initial fluid sample is flowed through the magnetic separator system 280 at the same flow rate of 0.8 mL/min to obtain the standard sample containing the first mixture of biological objects without further static magnetic sorting (i.e., one-pass-only sorting).

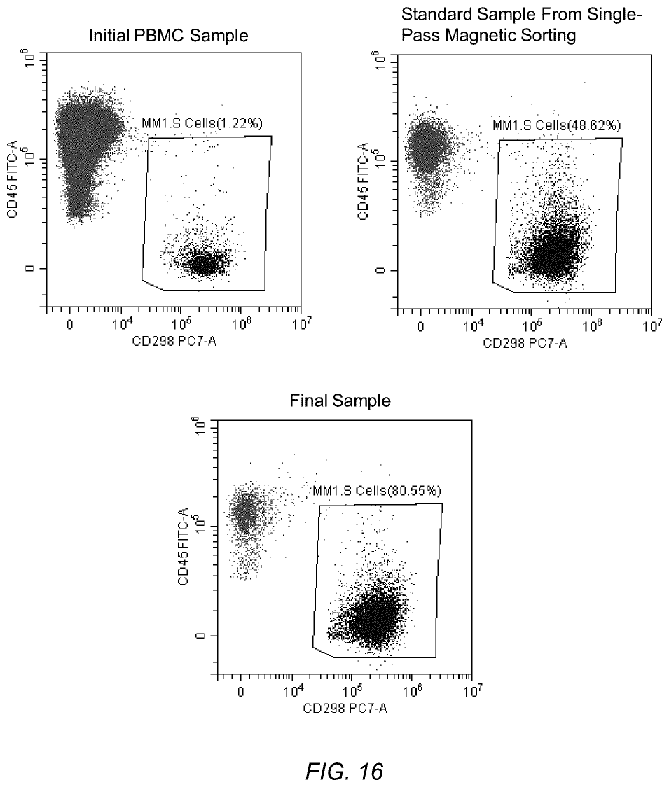

The compositions of the initial fluid sample, standard sample, and final sample, as measured by the flow cytometer (CytoFlex, Beckman Coulter) are shown in the dot plots of FIG. 16 and summarized in Table 2 below. The cytometry results show the final sample sorted in accordance with an embodiment of the present invention has a relatively high recovery ratio of 77.31% and 80.55% MM1S purity compared with 1.22% MM1S purity of the spiked PBMC sample. Compared with the standard process (single-pass-only sorting), which produces the standard sample, the current invention increases the MM1S purity from 48.62% to 80.55% with only slight decrease in the recovery ratio.

TABLE 2
MM1S cell purity and recovery ratio for initial PBMC, standard, and
final samples

| Sample | Purity | Recovery ² |
|---|---|---|
| Initial PBMC sample | 1.22% | |
| Standard sample (single-pass-only sorting) | 48.62% | 80.28% |
| Final sample (with additional static sorting) | 80.55% | 77.31% |

² Compared with initial PBMC sample

### Example 3: Direct Isolation of CD4+ and CD8+ T Cells from Whole Blood without Leukapheresis or Buffy Coat Extraction

The sample preparation process begins by adding a first reagent (NanoSep™ CD4 Beads, Beijing T&L Biological Technology) at a dosage of 40 μL per mL blood and a second reagent (NanoSep™ CD8 Beads, Beijing T&L Biological Technology) at a dosage of 20 L per mL blood to a whole blood sample to magnetically label the T cells. The sizes of the magnetic beads in both reagents are about 50 nm in diameter. The mixture is incubated for 40 min at room temperature while rocking on a mixer (Nutating Mixer, Labnet International). The magnetically labeled whole blood sample is further diluted with about 1 time the volume of buffer fluid (i.e., 2-fold dilution) and gently mixed by pipetting up and down to yield the initial fluid sample for magnetic sorting.

An aliquot of the initial whole blood sample is flowed through the magnetic separator system 280 at a flow rate of 0.5 mL/min to deposit a first mixture of biological objects on the conduit wall, after which the first mixture of biological objects is further sorted using the static magnetic sorting process described above, resulting in the second mixture of biological objects in the final sample. Another aliquot of the initial whole blood sample is flowed through the magnetic separator system 280 at the same flow rate of 0.5 mL/min to obtain the standard sample containing the first mixture of biological objects without further static magnetic sorting (i.e., one-pass-only sorting).

The compositions and the T-cell purity and recovery ratio for the initial whole blood, standard, and final samples, as measured by cytometry, are summarized in Table 3 below. The standard sample recovered after flowing the initial whole blood sample through the magnetic separator system 280 has 4.94% T-cell purity, which is a two-order improvement compared with 0.03% T-cell purity of the initial whole blood sample. Despite removal of 99.68% of the RBCs in the initial whole blood sample, the standard sample still contains about 94% RBCs, which is reflected by its pink appearance. The final sample recovered after the additional static sorting steps in accordance with an embodiment of the present invention has 43.95% T-cell purity, which is an order further improvement over the standard sample and a four-order improvement over the initial whole blood sample. The further improvement in the T-cell purity of the final sample over the standard sample is mainly due to the removal of additional RBCs by the additional static sorting: about 99.98% of RBCs in the initial whole blood sample is removed in the final sample, which is reflected by its clear appearance.

TABLE 3
Compositions and T-cell purity and recovery ratio for
initial whole blood, standard, and final samples

| | RBC | Platelet | Non-T | T-Cell | T-Cell | T-Cell | |
|---|---|---|---|---|---|---|---|
| Sample | Count | Count | WBC C. | Count | Purity | Recovery ³ | Color |
| Whole blood | 5.91 × 10⁹ | 4.95 × 10⁶ | 2.51 × 10⁶ | 1.74 × 10⁶ | 0.03% | | Red |
| Standard | 1.86 × 10⁷ | 9.05 × 10⁴ | 9.51 × 10⁴ | 9.78 × 10⁵ | 4.94% | 56.12% | Pink |
| Final sample | 1.18 × 10⁶ | 1.42 × 10³ | 6.67 × 10⁴ | 9.80 × 10⁵ | 43.95% | 56.25% | Clear |

³ Compared with initial whole blood sample

While the present invention has been shown and described with reference to certain preferred embodiments, it is to be understood that those skilled in the art will no doubt devise certain alterations and modifications thereto which nevertheless include the true spirit and scope of the present invention. Thus the scope of the invention should be determined by the appended claims and their legal equivalents, rather than by examples given.

Any element in a claim that does not explicitly state “means for” performing a specified function, or “step for” performing a specific function, is not to be interpreted as a “means” or “step” clause as specified in 35 U.S.C. § 112, ¶6. In particular, the use of “step of” in the claims herein is not intended to invoke the provisions of 35 U.S.C. § 112, ¶6.

What is claimed is:

1. A method for magnetically sorting biological objects comprising the steps of:
providing a fluid sample containing an initial biological mixture that includes magnetically labeled biological objects and unlabeled biological objects;
depositing a first biological mixture on a conduit wall of a conduit exposed to a magnetic field by flowing the fluid sample through the conduit;
rinsing the first biological mixture by flowing a stream of buffer fluid through the conduit and filling the conduit with the buffer fluid;
removing the magnetic field from the conduit;
resuspending the first biological mixture in the buffer fluid in the conduit to form a suspension of the first biological mixture by applying mechanical agitation to disperse the first biological mixture on the conduit wall into the buffer fluid; and
depositing a second biological mixture on the conduit wall by applying the magnetic field to the conduit while the suspension of the first biological mixture in the conduit remains stagnant,
wherein the second biological mixture has a magnetically labeled biological object purity higher than the first biological mixture.

2. The method of claim 1 further comprising the step of filling the conduit with an additional amount of the buffer fluid between the steps of removing the magnetic field from the conduit and resuspending the first biological mixture in the buffer fluid in the conduit.

3. The method of claim 1, wherein the step of resuspending the first biological mixture in the buffer fluid in the conduit is carried out by applying transverse vibration to the conduit while the suspension of the first biological mixture remains stagnant in the conduit.

4. The method of claim 3, wherein the transverse vibration is applied to the conduit by an arm clutching the conduit.

5. The method of claim 1 further comprising the steps of:
rinsing the second biological mixture by flowing another stream of buffer fluid through the conduit and filling the conduit with the buffer fluid while the magnetic field is applied to the conduit; and
removing the magnetic field from the conduit containing the second biological mixture.

6. The method of claim 5 further comprising the steps of:
resuspending the second biological mixture in the buffer fluid in the conduit to form a suspension of the second biological mixture by applying the mechanical agitation to disperse the second biological mixture on the conduit wall into the buffer fluid; and
depositing a third biological mixture on the conduit wall by applying the magnetic field to the conduit while the suspension of the second biological mixture in the conduit remains stagnant,
wherein the third biological mixture has a magnetically labeled biological object purity higher than the second biological mixture.

7. The method of claim 5 further comprising the step of eluting the second biological mixture with still another stream of buffer fluid through the conduit while the conduit is subjected to the mechanical agitation.

8. The method of claim 7, wherein the mechanical agitation is in a form of transverse vibration applied to the conduit.

9. The method of claim 1, wherein the steps of depositing the first and second biological mixtures on the conduit wall are performed by a same magnetic separator device.

10. The method of claim 1, wherein the magnetically labeled biological objects express CD34 antigen.

11. The method of claim 1, wherein the magnetically labeled biological objects express CD138 antigen.

12. The method of claim 1, wherein the magnetically labeled biological objects express CD4or CD8 antigen.

13. The method of claim 1, wherein the fluid sample includes whole blood.

14. The method of claim 13, wherein the magnetically labeled biological objects express CD4 or CD8 antigen.

15. The method of claim 13, wherein the magnetically labeled biological objects are magnetically labeled T cells.

16. The method of claim 15, wherein the second biological mixture has a T cell purity three orders higher than the initial biological mixture.

17. The method of claim 1, wherein the step of depositing a second biological mixture on the conduit wall is performed using a magnetic separator device comprising:
first and second permanent magnets each having first and second poles;
a center magnetic flux guide including a center tip having a tapering shape and a center base magnetically coupled to the first poles of the first and second permanent magnets;
a first side magnetic flux guide including a first side tip and a first side base magnetically coupled to the second pole of the first permanent magnet; and
a second side magnetic flux guide including a second side tip and a second side base magnetically coupled to the second pole of the second permanent magnet,
wherein the first and second side magnetic flux guides are disposed on opposite sides of the center magnetic flux guide with the first and second side tips positioned above the center tip and pointed at each other.

18. The method of claim 17, wherein the magnetically labeled biological objects include magnetic beads having a diameter of 50 nm.

19. The method of claim 17, wherein ends of the first and second side tips each have a chisel edge profile with a bevel side facing away from the center magnetic flux guide.

20. The method of claim 19, wherein the magnetic separator device further comprises a soft magnetic ridge structure pushing the conduit into a gap delineated by a tip end of the center tip and the bevel sides of the first and second side tips.
